(19) United States
(12) Patent Application Publication
(10) Pub. No.: US 2021/0068615 A1
(43) Pub. Date: Mar. 11, 2021

(54) ROBOTIC SURGICAL SYSTEM

(71) Applicant: Medrobotics Corporation, Raynham, MA (US)

(72) Inventors: Joseph M. Johnson, Norwood, MA (US); Paul Sinclair, Dearborn, MI (US); Mike A. Ciavaglia, Dearborn, MI (US); Ian J. Darisse, Southborough, MA (US); Nick Coussens, Ferndale, MI (US); Anthony Richard Polakowski, Avon Lake, OH (US); Anish Mampetta, Waterbeach Cambridgeshire (GB)

(21) Appl. No.: 16/960,162

(22) PCT Filed: Jan. 7, 2019
(86) PCT No.: PCT/US19/12484

§ 371 Date: Jul. 6, 2020

Related U.S. Application Data
(60) Provisional application No. 62/614,225, filed on Jan. 5, 2018.
(60) Provisional application No. 62/614,228, filed on Jan. 5, 2018.
(60) Provisional application No. 62/614,224, filed on Jan. 5, 2018.
(60) Provisional application No. 62/614,240, filed on Jan. 5, 2018.

(51) Int. Cl. A61B 1/008; A61B 1/018; A61B 1/005; A61B 1/00

(52) U.S. Cl. CPC A61B 1/008; A61B 1/018; A61B 1/0057; A61B 34/76; A61B 1/00149; A61B 1/00006; A61B 1/00133

(57) ABSTRACT

A system for performing a medical procedure on a patient includes an articulating probe assembly and at least one tool. The articulating probe assembly comprises an inner probe comprising multiple articulating inner links, an outer probe surrounding the inner probe and comprising multiple articulating outer links, and at least two working channels that exit a distal portion of the probe assembly. The at least one tool is configured to translate through one of the at least two working channels. A feeder controls the articulating probe assembly.

### RELATED APPLICATIONS

This application claims the benefit of U.S. Provisional Application No. 62/613,899, filed Jan. 5, 2018, the content of which is incorporated herein by reference in its entirety.

This application claims the benefit of U.S. Provisional Application No. 62/614,223, filed Jan. 5, 2018, the content of which is incorporated herein by reference in its entirety.

This application claims the benefit of U.S. Provisional Application No. 62/614,224, filed Jan. 5, 2018, the content of which is incorporated herein by reference in its entirety.

This application claims the benefit of U.S. Provisional Application No. 62/614,228, filed Jan. 5, 2018, the content of which is incorporated herein by reference in its entirety.

This application claims the benefit of U.S. Provisional Application No. 62/614,225, filed Jan. 5, 2018, the content of which is incorporated herein by reference in its entirety.

This application claims the benefit of U.S. Provisional Application No. 62/614,240, filed Jan. 5, 2018, the content of which is incorporated herein by reference in its entirety.

This application claims the benefit of U.S. Provisional Application No. 62/614,235, filed Jan. 5, 2018, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, PCT Publication No. WO2015/102939, the content of which is incorporated herein by reference in its entirety.

This application is related to U.S. patent application Ser. No. 14/892,750, filed Nov. 20, 2015, U.S. Publication No. 2016/0256226, now U.S. Pat. No. 10,004,568 issued on Jun. 26, 2018, the content of which is incorporated herein by reference in its entirety.

This application is related to U.S. patent application Ser. No. 15/899,826, filed Feb. 20, 2018, U.S. Publication No. 2018/0250095 the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, PCT Publication No. WO2012/054829, the content of which is incorporated herein by reference in its entirety.

This application is related to U.S. patent application Ser. No. 13/880,525, filed Apr. 19, 2013, U.S. Publication No. 2014/0005683, now U.S. Pat. No. 8,992,421, issued on Mar. 31, 2015, the content of which is incorporated herein by reference in its entirety.

This application is related to U.S. patent application Ser. No. 14/587,166, filed Dec. 31, 2014, U.S. Publication No. 2015/0313449, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US2012/040414, filed Jun. 1, 2012, PCT Publication No. WO2012/167043, the content of which is incorporated herein by reference in its entirety.

This application is related to U.S. patent application Ser. No. 14/119,316, filed Nov. 21, 2013, U.S. Publication No. 2014/0094825, the content of which is incorporated herein by reference in its entirety.

This application is related to U.S. Provisional Application No. 62/504,175, filed May 10, 2017, the content of which is incorporated herein by reference in its entirety.

This application is related to PCT Application No. PCT/US2018/031774, filed May 9, 2018, PCT Publication No. WO2018/0020898, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, PCT Publication No. WO2012/078309, the content of which is incorporated herein by reference in its entirety.

This application is related to U.S. patent application Ser. No. 13/884,407, filed May 9, 2013, U.S. Publication No. 2014/0012288, now U.S. Pat. No. 9,649,163, issued on May 16, 2017, the content of which is incorporated herein by reference in its entirety.

This application is related to U.S. patent application Ser. No. 15/587,832, filed May 5, 2017, U.S. Publication No. 2018/0021095, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US2012/032279, filed Apr. 5, 2012, PCT Publication No. WO2012/138834, the content of which is incorporated herein by reference in its entirety.

This application is related to U.S. patent application Ser. No. 14/008,775, filed Sep. 30, 2013, U.S. Publication No. 2014/0046305, now U.S. Pat. No. 9,962,179, issued on May 8, 2018, the content of which is incorporated herein by reference in its entirety.

This application is related to U.S. patent application Ser. No. 14/944,665, filed Nov. 18, 2015, U.S. Publication No.: 2016/0066938, the content of which is incorporated herein by reference in its entirety.

This application is related to U.S. patent application Ser. No. 14/945,685, filed Nov. 19, 2015, U.S. Publication No. 2016/0066939, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US2012/054802, filed Sep. 12, 2012, PCT Publication No. WO2013/039999, the content of which is incorporated herein by reference in its entirety.

This application is related to U.S. patent application Ser. No. 14/343,915, filed Mar. 10, 2014, U.S. Publication No. 2014/0371764, now U.S. Pat. No. 9,757,856, issued on Sep. 12, 2017, the content of which is incorporated herein by reference in its entirety.

This application is related to U.S. patent application Ser. No. 15/064,043, filed Mar. 8, 2016, U.S. Publication No. 2016/0262840, now U.S. Pat. No. 9,572,628, issued on Feb. 21, 2017, the content of which is incorporated herein by reference in its entirety.

This application is related to U.S. patent application Ser. No. 15/684,268, filed Aug. 23, 2017, U.S. Publication No. 2017/0368681, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, PCT Publication No. WO2012/015659, the content of which is incorporated herein by reference in its entirety.

This application is related to U.S. patent application Ser. No. 13/812,324, filed Jan. 25, 2013, U.S. Publication No. 2014/0012287, now U.S. Pat. No. 9,901,410, issued on Feb. 27, 2018, the content of which is incorporated herein by reference in its entirety.

This application is related to U.S. patent application Ser. No. 15/874,189, filed Jan. 18, 2018, U.S. Publication No. 2018-0206923 the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US2012/070924, filed Dec. 20, 2012, PCT Publication No. WO2013/096610, the content of which is incorporated herein by reference in its entirety.

This application is related to U.S. patent application Ser. No. 14/364,195, filed Jun. 10, 2014, U.S. Publication No. 2014/0318299, now U.S. Pat. No. 9,364,955 issued on Jun. 14, 2016, the content of which is incorporated herein by reference in its entirety.

This application is related to U.S. patent application Ser. No. 15/180,503, filed Jun. 13, 2016, U.S. Publication No. 2017/0015007, now U.S. Pat. No. 9,821,477, issued on Nov. 21, 2017, the content of which is incorporated herein by reference in its entirety.

This application is related to U.S. patent application Ser. No. 15/786,901, filed Oct. 18, 2017, U.S. Publication No. 2018/0161992, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US2013/054326, filed Aug. 9, 2013, PCT Publication No. WO2014/026104, the content of which is incorporated herein by reference in its entirety.

This application is related to U.S. patent application Ser. No. 14/418,993, filed Feb. 2, 2015, U.S. Publication No. 2015/0282835, now U.S. Pat. No. 9,675,380 issued on Jun. 13, 2017, the content of which is incorporated herein by reference in its entirety.

This application is related to U.S. patent application Ser. No. 15/619,875, filed Jun. 12, 2017, U.S. Publication No. 2018/0021060, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US2014/010808, filed Jan. 9, 2014, PCT Publication No. WO2014/110218, the content of which is incorporated herein by reference in its entirety.

This application is related to U.S. patent application Ser. No. 14/759,020, filed Jul. 2, 2015, U.S. Publication No. 2015/0342690, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US2013/043858, filed Jun. 3, 2013, PCT Publication No. WO2013/184560, the content of which is incorporated herein by reference in its entirety.

This application is related to U.S. patent application Ser. No. 14/402,224, filed Nov. 19, 2014, U.S. Publication No. 2015/0150633, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US2013/038701, filed May 20, 2014, PCT Publication No. WO2014/189876, the content of which is incorporated herein by reference in its entirety.

This application is related to U.S. patent application Ser. No. 14/888,541, filed Nov. 2, 2015, U.S. Publication No. 2016/0074028, now U.S. Pat. No. 9,517,059, issued on Dec. 13, 2016, the content of which is incorporated herein by reference in its entirety.

This application is related to U.S. patent application Ser. No. 15/350,549, filed Nov. 14, 2016, U.S. Publication No. 2017/0119364, now U.S. Pat. No. 10,016,187, issued on Jul. 10, 2018, the content of which is incorporated herein by reference in its entirety.

This application is related to U.S. patent application Ser. No. 16/020,115, filed Jun. 27, 2018, U.S. Publication No. 2018/0368823, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US2014/036571, filed May 2, 2014, PCT Publication No. WO2014/179683, the content of which is incorporated herein by reference in its entirety.

This application is related to U.S. patent application Ser. No. 14/888,189, filed Oct. 30, 2015, U.S. Publication No. 2016/0067000, now U.S. Pat. No. 9,913,695, issued on Mar. 13, 2018, the content of which is incorporated herein by reference in its entirety.

This application is related to U.S. patent application Ser. No. 15/916,664, filed Mar. 9, 2018, U.S. Publication No. 2018/0256269, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US2014/067091, filed Nov. 24, 2014, PCT Publication No. WO2015/081008, the content of which is incorporated herein by reference in its entirety.

This application is related to U.S. patent application Ser. No. 15/038,531, filed May 23, 2016, U.S. Publication No. 2016/0287224, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US2015/034424, filed Jun. 5, 2015, PCT Publication No. WO2015/188071, the content of which is incorporated herein by reference in its entirety.

This application is related to U.S. patent application Ser. No. 15/315,868, filed Dec. 2, 2016, U.S. Publication No. 2017/0100197, the content of which is incorporated herein by reference in its entirety.

This application is related to U.S. patent application Ser. No. 16/225,156, filed Dec. 19, 2018, U.S. Publication No. 2019/______, the content of which is incorporated herein by reference in its entirety.

This application is related to U.S. Provisional Application No. 62/150,223, filed Apr. 20, 2015, the content of which is incorporated herein by reference in its entirety.

This application is related to U.S. Provisional Application No. 62/299,249, filed Feb. 24, 2016, the content of which is incorporated herein by reference in its entirety.

This application is related to PCT Application No. PCT/US2016/028374, filed Apr. 20, 2016, PCT Publication No. WO2016/172162, the content of which is incorporated herein by reference in its entirety.

This application is related to U.S. patent application Ser. No. 15/567,109, filed Oct. 17, 2017, U.S. Publication No. 2018-0228557 the content of which is incorporated herein by reference in its entirety.

This application is related to U.S. Provisional Application No. 62/401,390, filed Sep. 29, 2016, the content of which is incorporated herein by reference in its entirety.

This application is related to PCT Application No. PCT/US2017/054297, filed Sep. 29, 2017, PCT Publication No. WO2018/064475, the content of which is incorporated herein by reference in its entirety.

This application is related to U.S. Provisional Application No. 62/517,433, filed Jun. 9, 2017, the content of which is incorporated herein by reference in its entirety.

This application is related to PCT Application No. PCT/US2018/036876, filed Jun. 11, 2018, PCT Publication No. WO2018/227180, the content of which is incorporated herein by reference in its entirety.

This application is related to U.S. Provisional Application No. 62/481,309, filed Apr. 4, 2017, the content of which is incorporated herein by reference in its entirety.

This application is related to U.S. Provisional Application No. 62/598,812, filed Dec. 14, 2017, the content of which is incorporated herein by reference in its entirety.

This application is related to U.S. Provisional Application No. 62/617,513, filed Jan. 15, 2018, the content of which is incorporated herein by reference in its entirety.

This application is related to PCT Application No. PCT/US2018/026016, filed Apr. 4, 2018, PCT Publication No. WO2018/187425 the content of which is incorporated herein by reference in its entirety.

This application is related to U.S. Provisional Application No. 62/533,644, filed Jul. 17, 2017, the content of which is incorporated herein by reference in its entirety.

This application is related to U.S. Provisional Application No. 62/614,263, filed Jan. 5, 2018, the content of which is incorporated herein by reference in its entirety.

This application is related to PCT Application No. PCT/US2018/042449, filed Jul. 17, 2018, PCT Publication No. WO2019/______, the content of which is incorporated herein by reference in its entirety.

This application is related to U.S. Provisional Application No. 62/582,283, filed Nov. 6, 2017, the content of which is incorporated herein by reference in its entirety.

This application is related to U.S. Provisional Application No. 62/614,346, filed Jan. 5, 2018, the content of which is incorporated herein by reference in its entirety.

This application is related to PCT Application No. PCT/US2018/059338, filed Nov. 6, 2018, PCT Publication No. WO2019/______, the content of which is incorporated herein by reference in its entirety.

This application is related to U.S. Design application No. 29/632,148, filed Jan. 5, 2018, the content of which is incorporated herein by reference in its entirety.

This application is related to U.S. Pat. No. 9,011,318, issued Apr. 21, 2015, the content of which is incorporated herein by reference in its entirety.

### BACKGROUND

As less invasive medical techniques and procedures become more widespread, medical professionals such as surgeons may require articulating surgical tools, such as endoscopes, to perform such less invasive medical techniques and procedures that require access to locations within the patient, such as a site accessible through the mouth or other natural orifice, or a site accessible through an incision through the patient's skin.

There is a need for improved systems for performing a medical procedure.

### SUMMARY

In an aspect, a system for performing a medical procedure on a patient, comprises: an articulating probe assembly, comprising: an inner probe comprising multiple articulating inner links; an outer probe surrounding some or all of the inner probe and comprising multiple articulating outer links; and at least two working channels that exit a distal portion of the probe assembly; at least one tool configured to translate through one of the at least two working channels; and a feeder for controlling the articulating probe assembly.

In an embodiment, the articulating probe assembly comprises: an inner distal tip; at least one inner cable extending through the at least two working channels; and a clearance between the inner distal tip and the at least one inner cable.

In an embodiment, the system further comprises four inner cables for increasing a payload.

In an embodiment, at least one of the links comprises: four working channels in four separate lumens formed by a combination of inner and outer segments of the links; a sphere on a cone interface; proximal and distal steering sections; four groups of three holes around a diameter of an outer probe; a plurality of hourglass-shaped holes for a cable and working channel clearance; and a large cable clearance for decreased friction.

In an embodiment, the system further comprises a plurality of small diameter swaged tungsten cables to reduce friction.

In an embodiment, the system further comprises a plurality of torque transmitting working channels that prevent probe rotation.

In an embodiment, the system further comprising a funnel tip at the proximal end of the working channels, wherein the added friction between the tip and a support assembly prevents the working channels from rotating.

In an embodiment, the mating faces of the inner segments are sandblasted, including concave and convex spheres for high friction to increase a payload.

In an embodiment, inner surfaces that contact the working channels are polished to reduce friction.

In an embodiment, a dry lubricant is applied to an inner diameter and outer diameter of the working channels to reduce friction at the instrument and inner segments.

In an embodiment, the system includes a support assembly that includes a removable section, allowing the working channel to be removed and straightened to allow a passage of a long rigid camera connector.

In an embodiment, the system further comprises a long straight introduction device that supports a probe and allows a clearance between a patient and a robot.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same elements throughout the different views. The drawings are not necessarily to scale, emphasis instead being placed upon illustrating the principles of the preferred embodiments.

FIG. 1 is a schematic view of a system in which embodiments of the present inventive concepts can be practiced.

FIGS. 1A-C are graphic demonstrations of a robotic probe, in accordance with embodiments of the present inventive concepts.

FIG. 2 is a perspective view of a base of a manipulation assembly of a robotic apparatus in accordance with embodiments of the present inventive concepts.

FIGS. 3A and 3B are perspective top and bottom views, respectively, of a portion of a carriage of a robotic apparatus in accordance with embodiments of the present inventive concepts.

FIG. 4 is a perspective view of the carriage of the manipulation assembly of FIGS. 3A and 3B operably attached to base of FIG. 2.

FIG. 4A is a perspective view of a probe support assembly and probe translation assembly of a manipulation assembly attached to the carriage of FIG. 3 in accordance with embodiments of the present inventive concepts.

FIG. 4B is a perspective view of the carriage of FIGS. 3-4A operably attached to base of FIG. 2 in accordance with embodiments of the present inventive concepts.

FIGS. 5A-5C are views of a link of a probe in accordance with embodiments of the present inventive concepts.

FIG. 6A includes views of an outer link of a probe in accordance with embodiments of the present inventive concepts.

FIG. 6B includes views of an inner link of a probe in accordance with embodiments of the present inventive concepts.

FIG. 7 is perspective partial cut away view of a distal portion of a probe in accordance with embodiments of the present inventive concepts.

FIGS. 8A-8C are views of various links of a probe in accordance with embodiments of the present inventive concepts.

FIG. 9 is a perspective view of a probe assembly in accordance with embodiments of the present inventive concepts.

FIGS. 9A and 9B are perspective views of an underside of the probe assembly of FIG. 9.

FIG. 10 is a perspective view of an underside of a manipulation assembly of the probe assembly of FIG. 9 in accordance with embodiments of the present inventive concepts.

FIGS. 11 and 12 are sectional views of portions of the manipulation assembly of FIGS. 9-10 in accordance with embodiments of the present inventive concepts.

FIGS. 13A and B are perspective and bottom views of portions of a casing in accordance with embodiments of the present inventive concepts.

FIG. 14A is a perspective view of a bobbin 376 operably attached to a casing in accordance with embodiments of the present inventive concepts.

FIG. 14B is a sectional view of two bobbins in accordance with embodiments of the present inventive concepts.

### DETAILED DESCRIPTION OF EMBODIMENTS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate” shall include locations relatively close to, on, in and/or within a referenced component, anatomical location, or other location.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

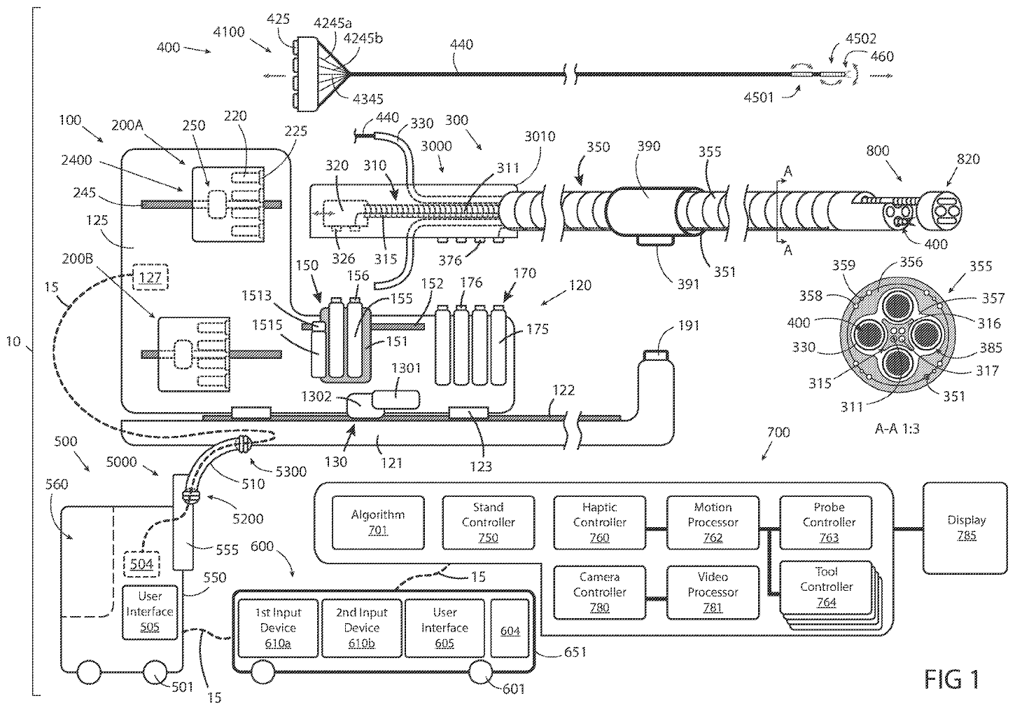

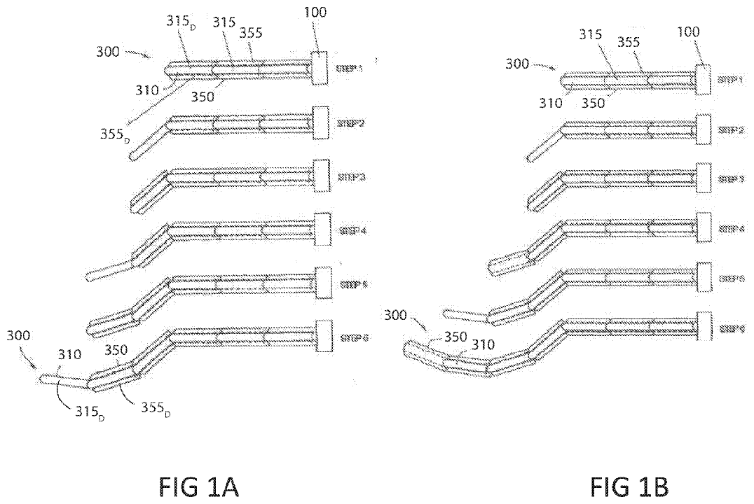

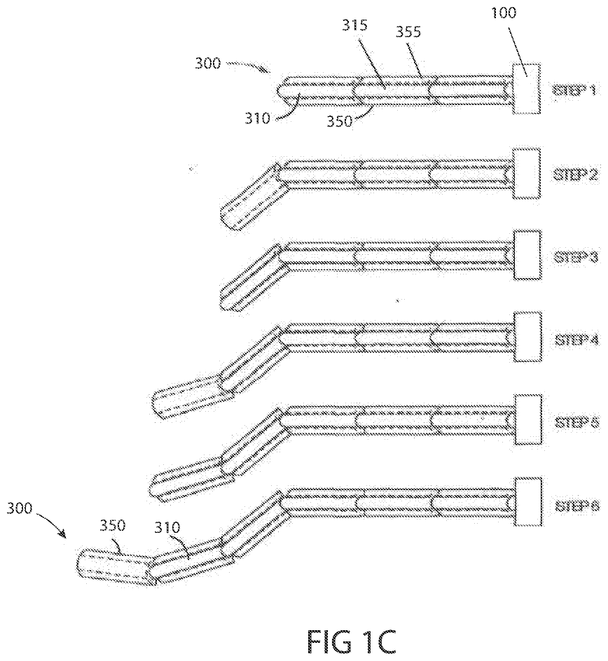

Referring to FIG. 1, a schematic view of a system in which embodiments of the present inventive concepts can be practiced is illustrated.

System 10 includes a robotic feeder 100. Feeder 100 interchangeably and operably engages a robotic probe assembly 300, and at least one robotic tool assembly 400. Feeder 100 is constructed and arranged to advance, retract, steer, and/or otherwise control the position and/or articulation of probe assembly 300 and/or tools 400, as described herein. One or more tools 400 can be slidingly received within a channel of probe assembly 300, and each tool 400 can be advanced beyond the distal end of probe assembly 300. Feeder 100 includes a probe manipulation assembly 120 for operably controlling the position and articulation of probe assembly 300. Feeder 100 also includes at least one tool manipulation assembly, tool drive 200 (e.g. tool drives 200A and 200B shown), for controlling the position and articulation of an attached tool 400. System 10 further includes a multi-dimensional positioning assembly, stand 500. Stand 500 includes an articulation assembly 5000 for positioning feeder 100 with multiple degrees of freedom, for example within an operating room, relative to a patient and/or patient bed, as described herein. System 10 further includes a control interface, surgeon console 600, configured to receive commands from one or more operators of system 10 (e.g. one or more surgeons or other clinicians). Console 600 can include a first and second input device, 610A and 610B respectively (singly or collectively input devices 610 herein), each configured to receive multi-dimensional input data (e.g. via a kinematic input device as described herein). System 10 further includes a collection of data processing components, collectively processing unit 700. Processing unit 700 can include one or more algorithms, controllers, memory, state machines, and/or processors, singly and/or collectively controlling one or more components of system 10 (e.g. based at least on one or more user inputs received by one or more input components of system 10). System 10 further includes an imaging device, camera assembly 800 (e.g. a tool 400 configured as a camera, as described herein), comprising one or more cameras, camera 820. Image data (e.g. still and/or video images) captured by camera 820 can be displayed on one or more monitors or other screens, display 785. One or more components described herein as included in a tool 400 can also be included in camera assembly 800, for example camera assembly 800 can comprise a tool 400 with camera 820 operably attached thereto. A conduit, bus 15, can connect one or more components of system 10. Bus 15 can comprise one or more electrical, fluid, optical, and/or other conduits for transferring information, power, one or more fluids, light energy, and combinations of one or more of these.

### Probe Assembly 300

Probe assembly 300 includes an outer probe 350, comprising multiple articulating outer links 355. Links 355 each comprise a ring-like structure (e.g. a hollow tube-like structure), link body 356, surrounding a hollow bore, channel 357. Collectively, channels 357 define a lumen extending along at least a portion of the length of outer probe 350. Links 355 can include multiple lumens extending therethrough, such as lumens extending along the link, through link body 356. For example, links 355 can include one or more steering cable lumens, lumens 358, such as eight lumens 358 shown. Lumens 358 can each slidingly receive a steering cable 351 that is used to control at least the articulation of outer probe 350, as described herein. Links 355 can also include one or more auxiliary lumens, four lumens 359 shown. In some embodiments, lumens 359 can slidingly receive elongate devices and/or filaments, such as optical fibers for delivering light to a surgical site.

Probe assembly 300 further includes inner probe 310, comprising multiple articulating inner links 315. Inner probe 310 is slidingly received within channels 356 extending through outer probe 350. Links 315 can comprise a link body 316, and can include multiple lumens extending therethrough, such as lumens extending along the link. For example, links 315 can include one or more steering cable lumens, lumens 317, such as four lumens 318 shown. Lumens 317 can each slidingly receive a steering cable 311 used to control at least the articulation of inner probe 310, as described herein.

The outer shape of link body 316 can align with the shape of the channel 357 to form a plurality of passageways or working channels 385, extending throughout probe assembly 300. Working conduits 330 can be slidingly received within channels 385, extending throughout the probe assembly 300. Each conduit 330 can sliding receive at least a portion of a tool 400.

Probe assembly 300 can be of similar construction and arrangement to the similar device described in reference to applicant's co-pending U.S. patent application Ser. No. 16/114,681, filed Aug. 28, 2018, the content of which is incorporated herein by reference in its entirety.

Probe assembly 300 further comprises a manipulation assembly 3000, operably attached to the proximal portion of probes 310, 350. Manipulation assembly 3000 comprises a housing 3010, surrounding at least a cart 320, operably attached to inner probe 310. Manipulation assembly 3000 comprises one or more bobbins 376 operably attached to one or more steering cables 351 (also referred to herein as control cables). Cart 320 comprises one or more bobbins 326 operably attached to one or more steering cables 311. Manipulation assembly 3000 is constructed and arranged to operably and removably attach to feeder 100, as described herein. Manipulation assembly 3000 supports the proximal sections of one or more working conduits 330 in an orientation that is radially dispersed relative to the radially compact orientation of the distal portions of working conduits 330 within probe assembly 300.

Probe assembly 300 can include a support structure, introducer 390. Introducer 390 can comprise a rigid elongate structure. Introducer 390 can surround at least a portion of probe assembly 300. Introducer 390 can comprise a connector portion 391, constructed and arranged to operably attach to a portion of feeder 100 as described herebelow.

Probe assembly 300 can be of similar construction and arrangement to the similar device described in applicant's co-pending application U.S. Provisional Application No. 62/614,240, filed Jan. 5, 2018, the content of which is incorporated herein by reference in its entirety.

### Feeder 100

Feeder 100 comprises a manipulation assembly 120 comprising a carriage 125 operably attached to a base 121. Carriage 125 can comprise one or more linear bearings 123 fixedly attached thereto, slidingly attached to a linear rail assembly 122, which in turn is fixedly attached to base 121. Linear rail assembly 122 can comprise one or more rails and/or lead screws. Manipulation assembly 120 can comprise a linear drive assembly 130, that is operably attached to carriage 125 and linear rail assembly 122. For example, linear rail assembly 122 can comprise at least a lead screw, and linear drive assembly 130 can comprise a motor 1301 and gear box 1302. Linear drive assembly 130 can be configured to engage the lead screw of linear rail assembly 122, such as to translate carriage 125 relative to base 121.

Manipulation assembly 120 can comprise a probe support assembly 170. Probe support assembly 170 can comprise at least a portion of carriage 125. Probe support assembly 170 can comprise one or more motors 175, each operably attached to a capstan 176. Probe support assembly 170 is constructed and arranged to operably and removably attach to manipulation assembly 3000, for example, such that each capstan 176 operably engages a corresponding bobbin 376. Motors 175 can be configured to rotate capstans 176, which in turn rotate bobbins 376, tensioning and de-tensioning cables 351 to control the articulation of outer probe 350.

Probe support assembly 170 can further comprise a probe translation assembly 150. Probe translation assembly 150 can comprise one or more motors 155, each operably attached to a capstan 156. Probe translation assembly 150 is constructed and arranged to operably and removably attach to cart 320, for example such that each capstan 156 operably engages a corresponding bobbin 326. Motors 155 can be configured to rotate capstans 156, which in turn rotate bobbins 326, tensioning and de-tensioning cables 311 to control the articulation of inner probe 310. Probe translation assembly 150 can comprise a cart 151. Motors 155 can be fixedly attached to cart 151. Cart 151 can be slidingly attached to a linear rail assembly 152, fixedly attached to carriage 125. Linear rail assembly 152 can comprise one or more rails and/or lead screws. Probe translation assembly 150 can comprise a motor 1515 and drive gear 1513 operably attached thereto. Drive gear 1513 can operably attach to linear rail assembly 152, for example when linear rail assembly 152 comprises at least a lead screw. Motor 1515 can be configured to rotate drive gear 1513 to translate cart 151 relative to carriage 125. Cart 151 can be constructed and arranged to engage cart 320, such that translation of cart 151 causes the translation of cart 320 within manipulation assembly 3000. Translation of cart 320 can cause the translation of inner probe 310 with respect to outer probe 350, as described herein.

Feeder 100 can include a connector portion 191, constructed and arranged to removably connect to introducer 390 of probe assembly 300. Connector portion 191 can be positioned at the distal end of carriage 125, as shown.

Feeder 100 can include one or more modules 127, such as one or more processors and/or controllers. Module 127 can be operably attached to one or more components of system 10 via bus 15.

Feeder 100 can be of similar construction and arrangement to the similar device described in applicant's co-pending application U.S. Provisional Application No. 62/614,240, filed Jan. 5, 2018, the content of which is incorporated herein by reference in its entirety.

### Tool Drive 200

Each tool drive 200 (also referred to herein as a singular tool drive 200) is configured to operably and interchangeably attach to one or more tools 400. Feeder 100 can comprise one, two, three, four, or more tool drives, tool drives 200A and 200B shown. Additional tool drives can be mounted to carriage 125 opposite tool drives 200A and 200B (e.g. on the opposite side of carriage 125). Tool drive 200 can slidingly attach to carriage 125 via a translation assembly 2400. Translation assembly 2400 can comprise a linear rail assembly 245, fixedly attached to carriage 125. Linear rail assembly 245 can comprise one or more rails and/or lead screws. Translation assembly 2400 can further comprise a linear drive assembly 250, operably attached to tool drive 200 and linear rail assembly 245. For example, linear rail assembly 245 can comprise at least a lead screw, and linear drive assembly 250 can comprise a motor and/or a gear box. Linear drive assembly 250 can be configured to engage the lead screw of linear rail assembly 245, to translate tool drive 200 relative to carriage 125. Translation of tool drive 200 can cause the translation of an attached tool 400, for example relative to outer probe 350 operably attached to manipulation assembly 120.

Tool drive 200 can comprise one or more motors 220, configured to manipulate one or more components of tool drive 200. For example, one or more motors 220 can be configured to rotate one or more assemblies of tool drive 200 relative to each other, and/or to rotate one or more gears 225 (e.g. capstans) of tool drive 200. Gears 225 of tool drive 200 can be configured to operably engage one or more bobbins of an attached tool 400, as described herein, to control the articulation of the attached tool 400.

Tool drive 200 can be of similar construction and arrangement to the similar device described in applicant's co-pending application U.S. Provisional Application No. 62/614,228, filed Jan. 5, 2018, the content of which is incorporated herein by reference in its entirety.

### Tool 400

Tool 400 can include a manipulation assembly 4100, operably attached to the proximal end of a shaft 440. Shaft 440 can comprise a flexible shaft, comprising one or more lumens. Tool 400 can comprise one or more sets of steering (or control) cables 4245a, 4245b, and or 4345. Cables 4245a,b can be operably attached to manipulation assembly 4100, and extend through shaft 440 to a first and second articulation section 4501 and 4502, respectively. Cables 4245a,b can be tensioned and/or de-tensioned by manipulation assembly 4100 to cause the articulation of articulation sections 4501 and 4502, respectively. Cables 4345 can be operably attached to manipulation assembly 4100, and extend through shaft 440 to an end effector 460. Cables 4345 can be tensioned and/or de-tensioned by manipulation assembly 4100 to cause the articulation or other manipulation of end effector 460. System 10 can comprise multiple tools 400, such as four, five, six, or more tools 400, each exchangeable and operably attachable to tool drives 200. End effectors 460 can comprise scissors, graspers, blades, cautery devices, laser devices, and the like. Manipulation assembly 4100 can be constructed and arranged to removably attach to tool drive 200, such that gears 225 engage bobbins 425 of manipulation assembly 4100. Motors 220 of tool drive 200 can rotate gears 225, and bobbins 425, to tension and/or de-tension one or more cables of tool 400 described herein, to tension and/or de-tension the cables and manipulate tool 400. Manipulation assembly 4100 can also be constructed and arranged to rotate one or more components of tool 400 relative to each other, for example to rotate end effector 460 relative to shaft 440.

Tool 400 can be of similar construction and arrangement to the similar device described in applicant's co-pending application U.S. Provisional Application No. 62/614,225, filed Jan. 5, 2018, the content of which is incorporated herein by reference in its entirety.

### Camera Assembly 800

In some embodiments, as described hereabove, a tool 400 can be configured as a camera assembly 800. Camera assembly 800 can comprise a camera 820, operably attached to the distal end of shaft 440 of a tool 400. In some embodiments, camera 820 is attached to shaft 440 after shaft 440 has been inserted through probe assembly 300. For example, in some embodiments, camera 820 is larger than working channel 385.

Camera assembly 800 can be of similar construction and arrangement to the similar device described in applicant's co-pending application PCT International Patent Application No. PCT/US2018/059338, filed Nov. 6, 2018, the content of which is incorporated herein by reference in its entirety.

### Stand 500

Stand 500 can be constructed and arranged to position feeder 100 relative to a patient and/or patient bed, such as to position probe assembly 300 for a surgical procedure. For example, surgical procedures can include but are not limited to transabdominal procedures, transoral procedures, trans anal procedures, and/or trans vaginal procedures. Stand 500 includes a base 550, supporting an articulation assembly 5000. Articulation assembly 5000 includes a tower 555, extending vertically from base 550. A first hub 5200 is operably attached to tower 555. First hub 5200 can be adjusted along the height of tower 555, via one or more motors and/or vertical translation assemblies. First hub 5200 is operably attached to positioning arm 510, which is operably attached to a second hub 5300. Second hub 5300 is operably attached to base 121 of feeder 100. Hubs 5200 and 5300 can each comprise one or more motors, gears, hinges, axles, and the like, configured to manipulate the position of feeder 100 relative to stand 500. Bus 15 of system 10 can operably connect feeder 100 to stand 500. In some embodiments, bus 15 is routed through hubs 5200, 5300, arm 510, and/or tower 555, such that bus 15 is at least partially contained within articulation assembly 5000.

Stand 500 can comprise a recess 560. Articulation assembly 5000 can be configured to “fold” into a stowed position, with feeder 100 positioned at least partially within recess 560. In some embodiments stand 500 can comprise a processor 504 and a user interface 505. User interface 505 can include input and output functionality, such as a touchscreen monitor. User interface 505 can be configured to allow a user to control one or more components of system 10, for example the articulation of articulation assembly 5000. In some embodiments, stand 500 includes one or more wheels 501, and is constructed and arranged to be mobile. For example, stand 500 can be manually repositionable by a user and/or can be robotically repositionable, for example when wheels 501 are driven by one or more motors.

Stand 500 can be of similar construction and arrangement to the similar device described in applicant's co-pending application U.S. Provisional Application No. 62/614,223, filed Jan. 5, 2018, the content of which is incorporated herein by reference in its entirety.

### Surgeon Console 600

Surgeon console 600 can be operably attached to one or more components of system 10, such as via bus 15. Console 600 can comprise a base 651, supporting input devices 610a,b, and user interface 605. Console 600 can comprise a processor 604. In some embodiments, processor 604 can receive commands from input device 610a,b, and/or user interface 605. User interface 605 can be configured to allow a user to control one or more components of system 10. In some embodiments, user interface 605 can be a redundant interface of user interface 505, such that a user can perform the same operations from either interface. In some embodiments, console 600 includes one or more wheels 601, and is constructed and arranged to be mobile. For example, console 600 can be manually repositionable by a user and/or can be robotically repositionable, for example when wheels 601 are driven by one or more motors.

Console 600 can be of similar construction and arrangement to the similar device described in applicant's co-pending application U.S. Provisional Application No. 62/614,224, filed Jan. 5, 2018, the content of which is incorporated herein by reference in its entirety.

### Processor 700

Processing unit 700 can comprise one or more controllers and/or processors, located throughout system 10. For example, processor 700 can comprise a computer or other processing device, and/or can comprise one or more controllers or modules of system 10 (e.g. module 127 of feeder 100, processor 504 of stand 500, and/or processor 604 of user interface 600). Processing unit 700 can comprise one or more algorithms for processing data and/or commanding one or more components of system 10 to perform one or more operations. Processing unit 700 can comprise one or more controllers for controlling components of system 10. Processing unit 700 can comprise a stand controller 750, for operational control of stand 500. Processing unit 700 can comprise a camera controller, for operational control of camera assembly 800. Camera controller 780 can be operably attached to a video processor 781 for processing image data captured by camera 820. Video processor 781 can provide processed image data to a display 785, for display to a user. Processing unit 700 can comprise a haptic controller 760, operably attached to input devices 610a,b of console 600, for example via processor 604. Haptic controller 760 can be operably attached to a motion processor 762, which is operably attached to a probe controller 763, and one or more tool controllers 764. Haptic controller 760 can receive multi-dimensional input data (e.g. via a kinematic input device) from input devices 610a,b, and/or provide haptic feedback commands to input devices 610a,b. Motion processor 762 can process the multi-dimensional input data, and provide articulation and/or translation commands to probe controller 763 and/or tool controllers 764. Probe controller 763 can provide commands to one or more motors of system 10, for example to one or more motors of manipulation assembly 120 to at least advance, retract, steer, and/or otherwise control the position and/or articulation of probe assembly 300. Tool controllers 764 can provide commands to one or more motors of system 10, for example one or more motors of a tool drive 200 to at least advance, retract, steer, and/or otherwise control the position and/or articulation of an attached tool 400.

Processor 700 can be of similar construction and arrangement to the similar device described in applicant's co-pending application U.S. Provisional Application No. 62/614,235, filed Jan. 5, 2018, the content of which is incorporated herein by reference in its entirety.

Referring additionally to FIGS. 1A-C, graphic demonstrations of a robotic probe 300 are illustrated, consistent with the present inventive concepts. Articulating probe 300 comprises essentially two concentric mechanisms, an outer mechanism and an inner mechanism, each of which can be viewed as a steerable mechanism. Each of the components of probe 300 can comprise one or more sealing elements, such as to support an insufflation procedure. FIGS. 1A-C show the concept of how different embodiments of robotic probe 300 operate. Referring to FIG. TA, the inner mechanism can be referred to as a first mechanism or inner probe 310. The outer mechanism can be referred to as a second mechanism or outer probe 350. Each mechanism can alternate between rigid and limp states. In the rigid mode or state, the mechanism is just that—rigid. In the limp mode or state, the mechanism is highly flexible and thus either assumes the shape of its surroundings or can be re-shaped. It should be noted that the term “limp” as used herein does not necessarily denote a structure that passively assumes a particular configuration dependent upon gravity and the shape of its environment; rather, the “limp” structures described in this application are capable of assuming positions and configurations that are desired by the operator of the device, and therefore are articulated and controlled rather than flaccid and passive.

In some embodiments, one mechanism starts limp and the other starts rigid. For the sake of explanation, assume outer probe 350 is rigid and inner probe 310 is limp, as seen in step 1 in FIG. 1A. Now, inner probe 310 is both pushed forward by feeder 100, and a distal-most inner link 315D is steered, as seen in step 2 in FIG. 1A. Now, inner probe 310 is made rigid and outer probe 350 is made limp. Outer probe 350 is then pushed forward until a distal-most outer link 355D catches up to the distal-most inner link 315D (e.g. outer probe 350 is coextensive with inner probe 310), as seen in step 3 in FIG. 1A. Now, outer probe 350 is made rigid, inner probe 310 limp, and the procedure then repeats. One variation of this approach is to have outer probe 350 be steerable as well. The operation of such a device is illustrated in FIG. 1B. In FIG. 1B it is seen that each mechanism is capable of catching up to the other and then advancing one link beyond. According to one embodiment, outer probe 350 is steerable and inner probe 310 is not. The operation of such a device is shown in FIG. 1C.

In medical applications, operation, procedures, and so on, once robotic probe 300 arrives at a desired location, the operator, such as a surgeon, can slide one or more tools through one or more working channels of outer probe 350, inner probe 310, or one or more working channels formed between outer probe 350 and inner probe 310, such as to perform various diagnostic and/or therapeutic procedures. In some embodiments, the channel is referred to as a working channel that can, for example, extend between first recesses formed in a system of outer links and second recesses formed in a system of inner links. Working channels may be included on the periphery of robotic probe 300, such as working channels comprising one or more radial projections extending from outer probe 350, these projections including one or more holes sized to slidingly receive one or more tools. As described with reference to other embodiments, working channels may be positioned on other locations extending from, on, in, and/or within robotic probe 300.

Inner probe 310 and/or outer probe 350 are steerable and inner probe 310 and outer probe 350 can each be made both rigid and limp, allowing robotic probe 300 to drive anywhere in three-dimensions while being self-supporting. Articulating probe 300 can “remember” each of its previous configurations and for this reason, robotic probe 300 can retract from and/or retrace to anywhere in a three-dimensional volume such as the intracavity spaces in the body of a patient such as a human patient.

Inner probe 310 and outer probe 350 each include a series of links, i.e. inner links 315 and outer links 355 respectively, that articulate relative to each other. In some embodiments, outer links 355 are used to steer and lock robotic probe 300, while inner links 315 are used to lock robotic probe 300. In a “follow the leader” fashion, while inner links 315 are locked, outer links 355 are advanced beyond the distal-most inner link 315D. Outer links 355 are steered into position by the system steering cables, and then locked by locking the steering cables. The cable of inner links 315 is then released and inner links 315 are advanced to follow outer links 355. The procedure progresses in this manner until a desired position and orientation are achieved. The combined inner links 315 and outer links 355 may include working channels for temporary or permanent insertion of tools at the surgery site. In some embodiments, the tools can advance with the links during positioning of robotic probe 300. In some embodiments, the tools can be inserted through the links following positioning of robotic probe 300.

One or more outer links 355 can be advanced beyond the distal-most inner link 315D prior to the initiation of an operator controlled steering maneuver, such that the quantity extending beyond the distal-most inner link 315D will collectively articulate based on steering commands. Multiple link steering can be used to reduce procedure time, such as when the specificity of single link steering is not required. In some embodiments, between 2 and 20 outer links can be selected for simultaneous steering, such as between 2 and 10 outer links or between 2 and 7 outer links. The number of links used to steer corresponds to achievable steering paths, with smaller numbers enabling more specificity of curvature of robotic probe 300. In some embodiments, an operator can select the number of links used for steering (e.g. to select between 1 and 10 links to be advanced prior to each steering maneuver).

In some embodiments, a clearance between an inner distal tip and inner cables is provided to relieve cable torque and prevent inner twist.

In some embodiments, four cables at an inner region increases payload due to requirement for cables to elongate, and does not rely entirely on friction.

In some embodiments, a general segment design includes: four working channels contained in four separate lumens formed by inner and outer segments; sphere on cone interface; proximal and distal steering sections; four groups of three holes around diameter of outers, and in particular, eight for steering; four holes used for a light fiber operation; hourglass holes for cable and working channel clearance; and large cable clearance for decreased friction.

In some embodiments, small diameter swaged tungsten cables are used to reduce friction.

In some embodiments, torque transmitting working channels are used to prevent probe rotation. In some embodiments, a funnel tip is at the proximal end of the working channels. The added friction between the tip and support assembly prevents the working channels from rotating, thereby providing anti-rotation in the probe.

In some embodiments, sandblasting is performed on the mating faces of the inner segments (concave and convex spheres) for high friction to increase payload.

In some embodiments, polishing is performed on inner surfaces that contact the working channels to reduce friction.

In some embodiments, a dry lubricant is used on the ID and OD of the working channels to reduce friction at the instrument and inner segments.

In some embodiments, a removable section of a support assembly allows the working channel to be removed and straightened to allow passage of the long rigid camera connector.

In some embodiments, a long straight introducer supports the probe and allows clearance between a patient and a robot.

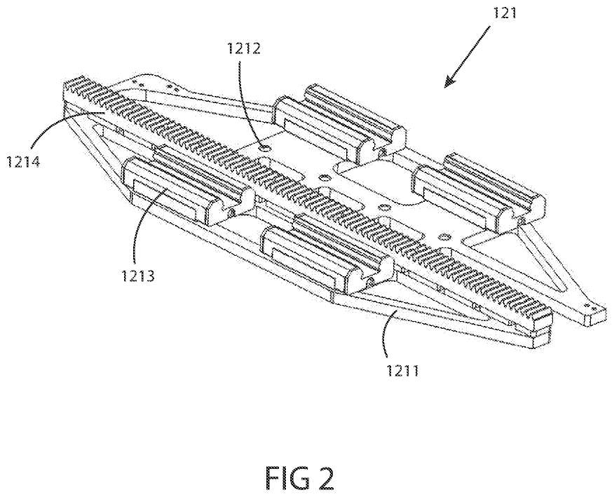

Referring to FIG. 2, a perspective view of a base 121 of a manipulation assembly 120 of a robotic apparatus is illustrated, in accordance with embodiments of the present inventive concepts.

Feeder 100 can comprise manipulation assembly 120 including base 121. In some embodiments, the base 121 includes a frame 1211 that is configured to fixedly attach to a support structure, for example articulation assembly 5000 of stand 500 shown and described herein. Frame 1211 can include one or more holes 1212 for mounting to a support structure. In some embodiments, the base 121 includes one or more (four shown) linear bearing carriages 1213 and a rack gear 1214.

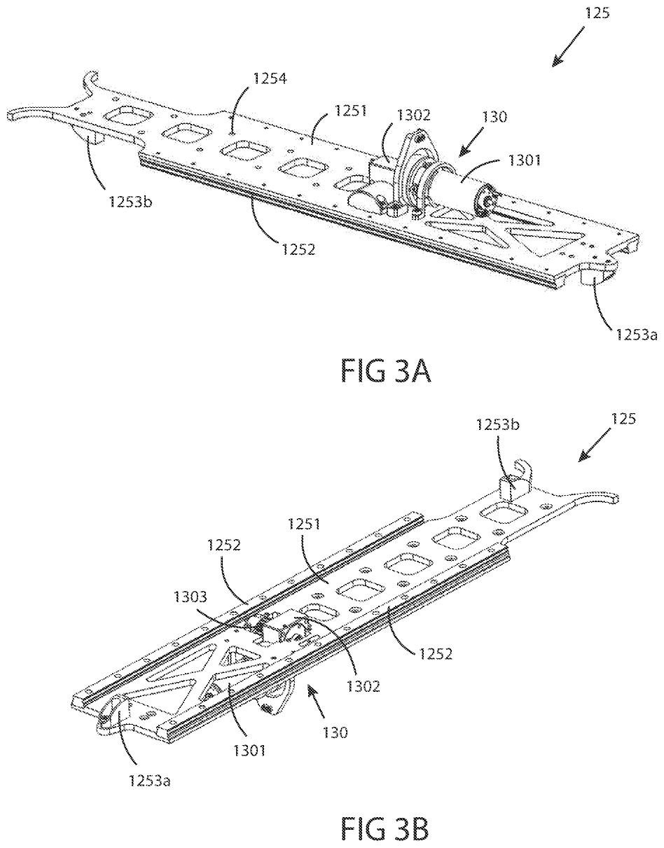

Referring to FIGS. 3A and B, perspective and bottom views of a portion of a carriage 125 of a robotic apparatus are illustrated, respectively, in accordance with embodiments of the present inventive concepts.

In some embodiments, the carriage 125 includes a base frame 1251 that is configured to slidingly attach to the frame 1211 of the base 121. In some embodiments, the carriage 125 includes one or more rails 1252, two of which are shown but not limited thereto, which extend along the length of the carriage 125 as shown, and are fixedly attached to the underside of the base frame 1251. The rails 1252 can be slidingly received by one or more of the linear bearing carriages 1213 of the base 121, thereby slidingly affixing the carriage 125 to the base 121, and limiting the relative motion between the base 121 and the carriage 125 along a length of the rails 1252.

In some embodiments, as shown in FIGS. 3A and 3B, a linear drive assembly 130 is fixedly attached to the base frame 1251. In some embodiments, the linear drive assembly 130 comprises a motor 1301 that is operably attached to a gear box 1302. In some embodiments, the gear box 1302 is operably attached to a pinion gear 1303 or the like. In some embodiments, the pinion gear 1303 extends through the base frame 1251, such that the pinion gear 1303 operably engages the rack gear 1214 of the base 121 shown in FIG. 2. The motor 1301 can drive the pinion gear 1303, causing the translation of the base frame 1251 along the rack gear 1214 and relative to the base 121. The gear box 1302 can comprise a non-back-drivable gear assembly, such that the position of the carriage 125 relative to the base 121 is relatively “locked” by the pinion gear 1303 and rack gear 1214, and is only adjustable by driving the motor 1301. In some embodiments, the gear ratios are configured to reduce noise and amperage. In some embodiments, the gear box 1302 is configured to reduce motor speed to under 3000 rpm to reduce noise.

The base frame 1251 can include one or more stoppers 1253, 1253a,b shown, on the proximal and distal ends of the base frame 1251 respectively. The stoppers 1253 can be constructed and arranged to abut the proximal and distal ends of frame 1211 of the base 121 to limit the travel of the base frame 1251 relative to the base 121.

In some embodiments, the tool drive 200 (not shown) comprises one or more stoppers 1253a,b on the proximal and distal end of the tool drive 200, constructed and arranged to limit proximal and distal travel of the tool drive 200. The length of travel can vary between two or more tool drives 200. The proximal travel of a tool drive 200 can be configured such that a tool 400 does not enter a patient during the installation of tool 400 into tool drive 200. In some embodiments, a sufficient clearance of tool drive 200 is configured to allow a tool 400 to be installed into tool drive 200 without an end effector 460 exiting a distal end of probe 300.

In some embodiments, the proximal travel of a tool drive 200 for use with a camera 820 is configured to prevent the camera 820 from being forcefully retracted against the distal end of the probe 300 (e.g. to prevent damage to and/or dislodgment of camera 820).

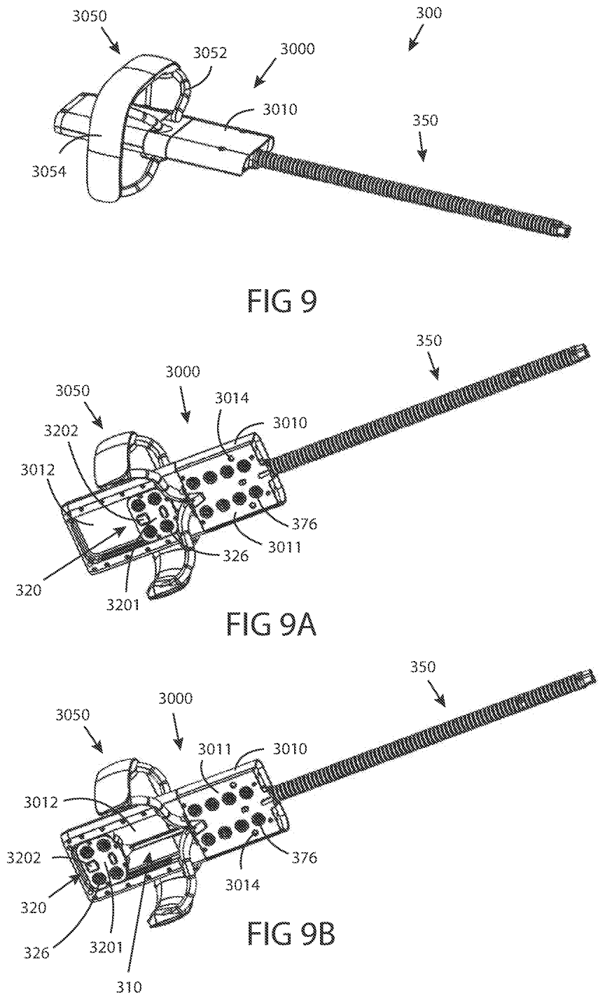

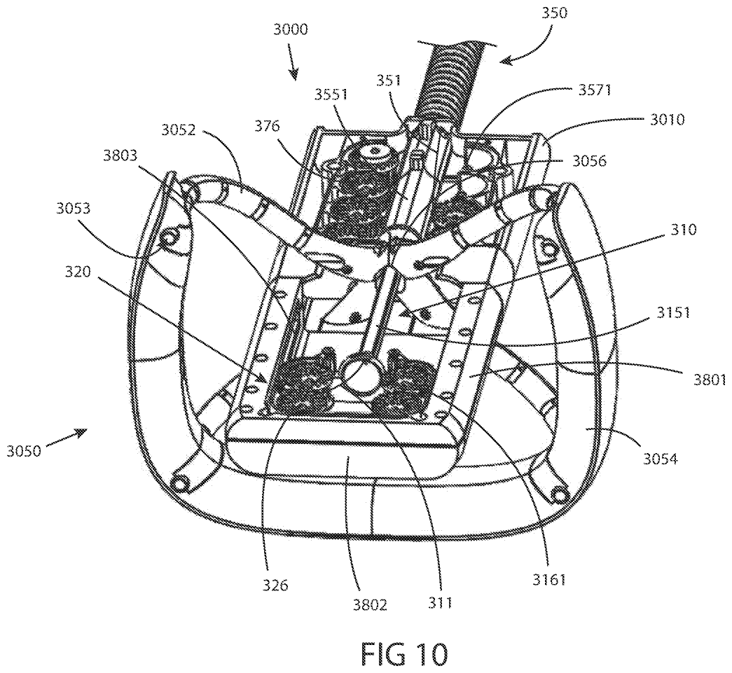

In some embodiments, the distal travel of tool drive 200 is configured to prevent collision with the support assembly 3050 as shown in FIG. 9.

In some embodiments, the base frame 1251 includes one or more mounting holes 1254 for attaching one or more additional components described herein.

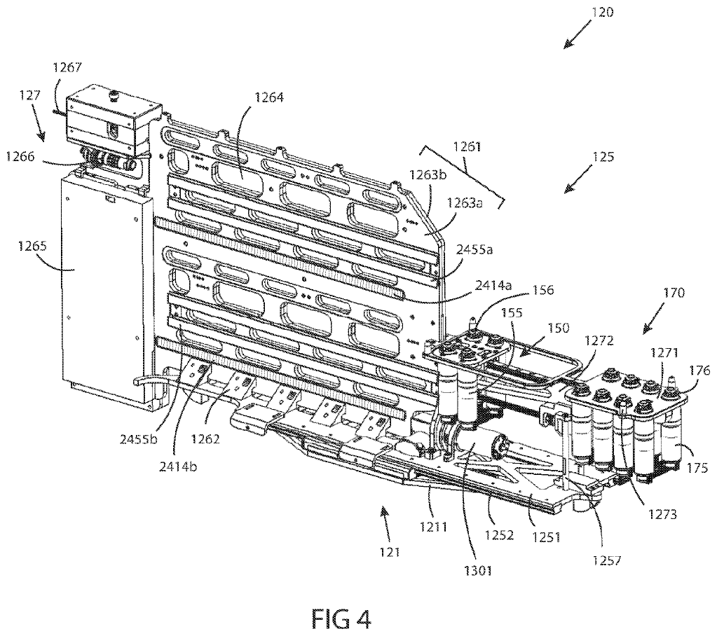

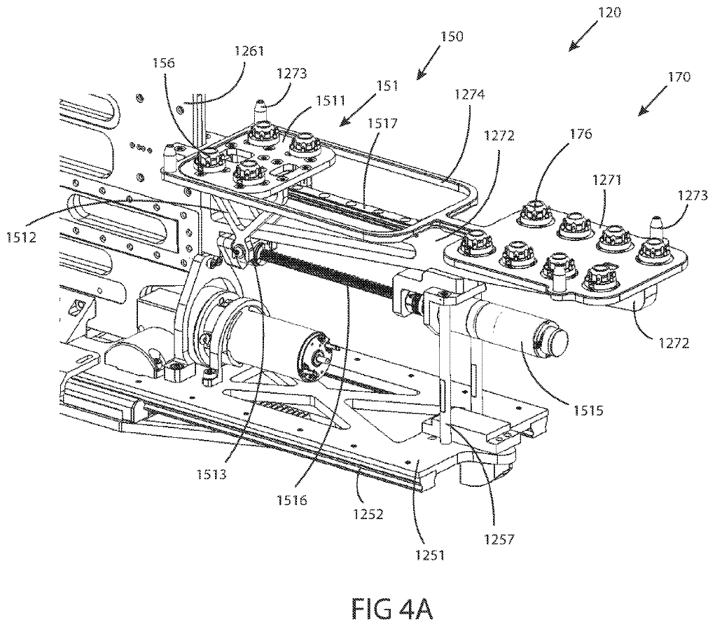

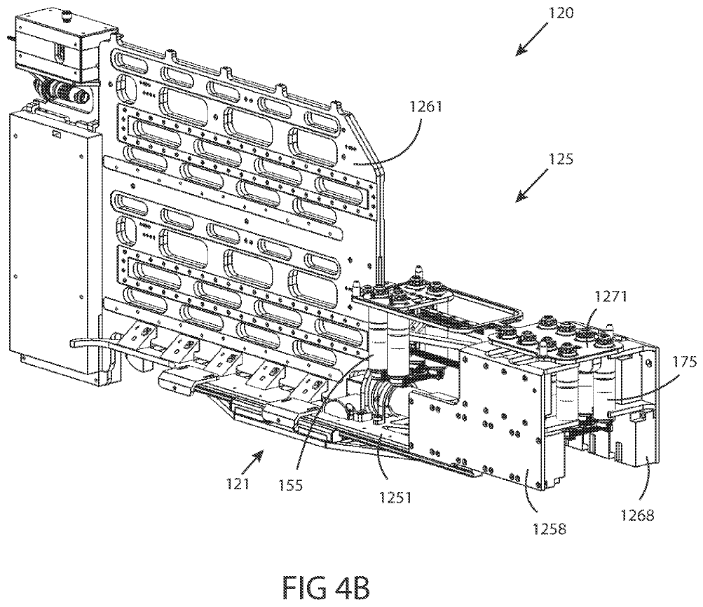

Referring to FIG. 4, a perspective view of a carriage 125 operably attached to a base 121 of a manipulation assembly 120 is illustrated, in accordance with embodiments of the present inventive concepts.

Carriage 125 can include a vertical frame 1261 that is fixedly attached to the base frame 1251. For example, the vertical frame 1261 is fixedly attached perpendicular to the base frame 1251. The vertical frame 1261 can be attached and/or supported with one or more braces 1262. Braces 1262 can include one or more conduits (not shown) for cable management. In some embodiments, as shown, the frame 1261 comprises two plates 1263a,b that are fixedly attached to be parallel to each other. In a manufacturing and/or an assembly process, the plates 1263a,b can be separated, such that one or more components can be fixedly attached to each plate 1263a,b in a mirrored fashion, e.g. in the same location on opposite sides of plates 1263a,b, using a matching screw or bolt pattern or other mechanical connector and recessed screws. The plates 1263a,b can then be fixedly attached to each other, with components affixed thereto without interference between attachment screws, and with matching placement and/or orientation on either side of the vertical frame 1261. For example, one or more tool drives 200 (not shown) can slidingly mount to the vertical frame 1261, slidingly attaching to rails 2455a,b as shown, and driven relative to linear rack gears 2414a,b as shown. The rails 2455a,b and racks 2414a,b can be fixedly attached to a plate 1263a with one or more screws attached from the inward side of the plate 1263a, when separated from the other plate 1263b.

In some embodiments, the vertical frame 1261 includes one or more cutouts 1264. The cutouts 1264 can be constructed and arranged to maximize airflow around the vertical frame 1261 (e.g. for cooling) and/or for weight reduction of the robotic apparatus. The vertical frame 1261 can support one or more modules 127, such as to support one or more processing units 1265, one or more connectors 1266, and/or one or more electrical buses 1267. A processing unit 1265 can be operably attached to one or more components of the carriage 125 (e.g. one or more motors) via bus 1267. Additionally or alternatively, connector 1266 can operably attach to a tool of the system 10 shown in FIG. 1, for example camera assembly 800, and can be operably attached to a processing unit 1265. The one or more processing units 1265 can be operably attached to a control unit of system 10, for example processing unit 700 and/or surgeon console 600 described in FIG. 1.

In some embodiments, the manipulation assembly 120 includes a probe support assembly 170 and an inner probe translation assembly 150, which can be fixedly attached to the proximal portion of the vertical frame 1261. The probe support assembly 170 can include one or more motors 175 for controlling the articulation of outer probe 350 as described herein, and inner probe translation assembly 150 includes one or more motors 155 for controlling the articulation and advancement of inner probe 310 as described herein.

Referring additionally to FIG. 4A, a perspective view of a probe translation assembly 150 and a probe support assembly 170 attached to the distal end of a vertical frame 1261 is illustrated, in accordance with embodiments of the present inventive concepts. Motors 175, 155 are not shown for illustrative clarity.

The probe support assembly 170 includes a bracket 1272 extending from the distal end of the vertical frame 1261. The bracket 1272 is constructed and arranged to support a frame 1271 of the probe support assembly 170. The probe support assembly frame 1271 supports one or more motors 175, extending below the frame 1271, each operably attached to a capstan 176. Each capstan 176 operably attaches to a bobbin 376 of probe assembly 300, as described herein. The base frame 1251 can include one or more standoffs 1257, supporting the distal portion of bracket 1272.

In some embodiments, the probe support assembly 1271 includes an open frame 1274, surrounding at least a portion of the probe translation assembly 150. In some embodiments, the probe translation assembly 150 includes a cart 151 comprising a frame 1511 and drive gear 1513, with connector 1512 therebetween to fixedly attach cart 151 to drive gear 1513. In some embodiments, connector 1512 comprises a U-shaped slot to allow for misalignment of cart 151 to drive gear 1513. Connector 1512 can be configured to avoid vertical tolerance stack. In some embodiments, the cart frame 1511 is slidingly attached to a rail 1517 (e.g. via cart 151). In some embodiments, a motor 1515 is operably attached to a lead screw 1516, operably engaged with drive gear 1513. The motor 1515 drives the lead screw 1516 to translate the cart 151 along the rail 1517. In some embodiments, the cart frame 1511 supports one or more motors 155, extending below frame 1511, each operably attached to a capstan 156. Each capstan 156 operably attaches to a bobbin 326 of probe assembly 300, as described herein. The probe translation assembly 150 operably attaches to cart 320 of inner probe 310, such that a translation of the cart 151 causes the translation of cart 320.

In some embodiments, an alignment pin 1273 operably engages a portion of manipulation assembly 3000, as described herebelow in reference to FIG. 9.

Referring additionally to FIG. 4B, a perspective view of a carriage 125 operably attached to a base 121 is illustrated, in accordance with embodiments of the present inventive concepts. One or more frames 1258 can be fixedly attached to the distal portion of the frame 1251. The frames 1258 can provide additional support to frame 1271, and can surround motors 175. The frames 1258 can also surround and support one or more motor controllers 1268 which can control one or more motors of manipulation assembly 120.

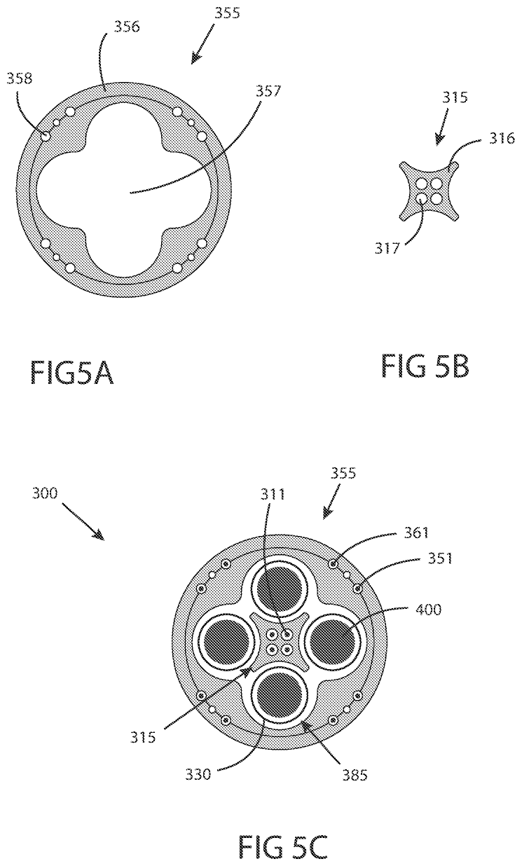

Referring to FIGS. 5A-C, end views of outer link 355, inner link 315, and a sectional view of probe 300 are illustrated, respectively, in accordance with embodiments of the present inventive concepts.

As described in reference to FIG. 1, the link 355 can comprise a ring-like structure, link body 356, surrounding a hollow bore, channel 357. The outer link 355 can include multiple lumens extending through link body 356. A lumen can include one or more steering cable lumens, for example, eight lumens 358 as shown.

Inner links 315 can comprise a link body 316, and include multiple lumens extending therethrough. A lumen can include one or more steering cable lumens 317, for example, four as shown.

As shown in FIG. 5C, the inner link 315 can be inserted and fit within a channel 357 of the outer link 355. The outer shape of 316 can align with the shape of the channel 357 to form a plurality of passageways or working channels 385, extending throughout probe 300. Working conduits 330 can be slidingly received within channels 385, extending throughout the probe 300. Each conduit 330 can slidingly receive at least a portion of a tool 400, as described hereabove in reference to FIG. 1. Conduits 330 can be constructed to transmit torque. Conduits 330 can be rotatably fixed to the distal tip of probe 300 to prevent rotation of probe 300.

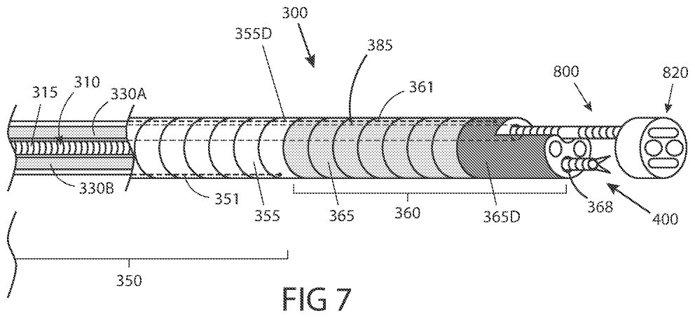

In some embodiments, one or more steering cables 351 and 311 can extend through lumens 358 and 317, respectively. Distal steering cables 361 can also extend through lumens 358, as described herebelow in reference to FIG. 7. Steering cables 351, 311 can flex (e.g. stretch) to accommodate a break and/or change in shape of probe 300.

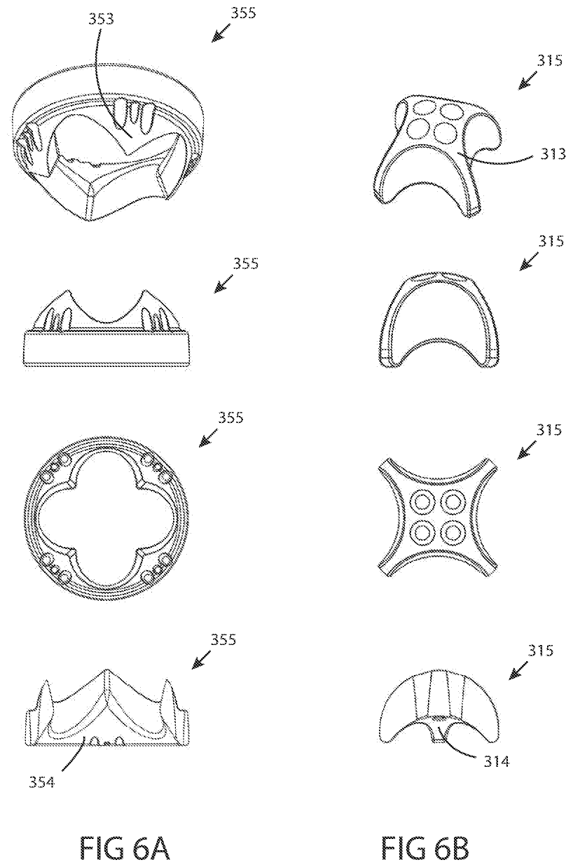

Referring to FIGS. 6A-B, a perspective view, a side view, a top view, and a sectional view of an outer link 355 and inner link 315 are illustrated, respectively, in accordance with embodiments of the present inventive concepts.

In some embodiments, outer links 355 each include a convex (e.g. spherical) articulating surface 353 and a concave (e.g. conical) articulating surface 354, as shown. Each outer link 355 can articulate relative to adjacent links 355, with surfaces 353 slidingly engaging surfaces 354.

In some embodiments, the inner links 315 each include a convex articulating surface 313, and a concave articulating surface 314, as shown. Each link 315 can articulate relative to adjacent links 315, with surfaces 313 slidingly engaging surfaces 314.

Referring to FIG. 7, a perspective partial cut away view of a distal portion of a probe 300 is illustrated, in accordance with embodiments of the present inventive concepts.

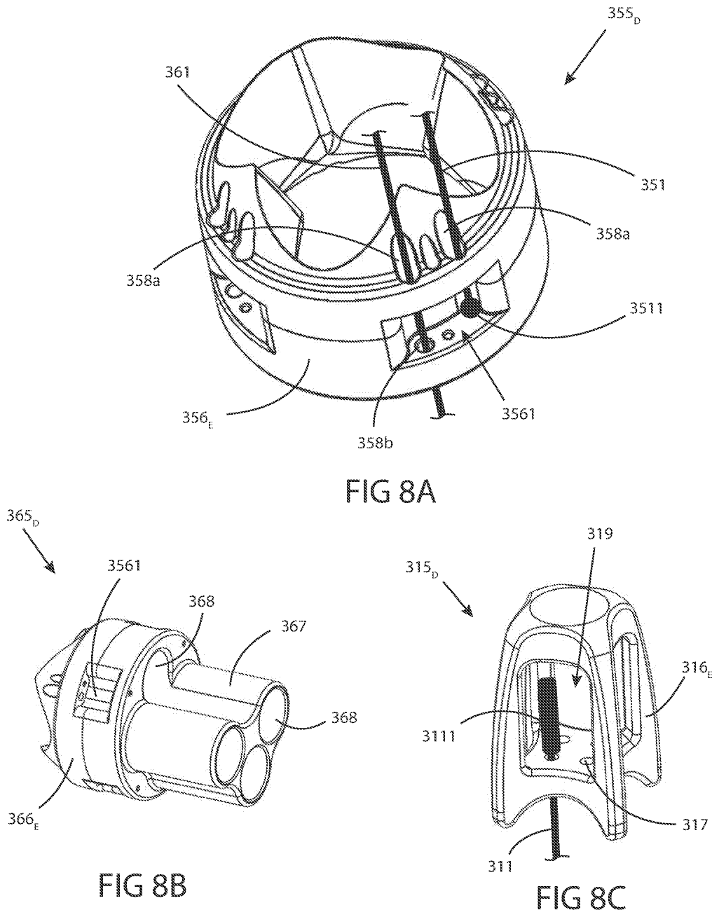

In some embodiments, the probe 300 comprises a set of distal links 365 that define a distal articulation section 360. The distal links 365 can be of similar construction and arrangement to outer links 355, so details thereof are not repeated due to brevity. In some embodiments, a first set of steering cables 351 extend through lumens 358 of links 355, and terminate at a distal most link 355, link 355D. Details of link 355D described herebelow in reference to FIG. 8A.

In some embodiments, a second set of steering cables 361 extend through lumens 358 of the outer links 355 (e.g., a first set in four lumens 385, a second set in four other lumens 358, eight lumens total, shown in FIG. 5C). In some embodiments, steering cables 361 extend beyond outer link 355D, through lumens 358 of links 365, and terminate at distal outer link 365D. Details of link 365D described herebelow in reference to FIG. 8B.

In some embodiments, the inner probe 310 is slidingly received within outer probe 350, along with one or more working conduits 330a,b (generally, 330) which are slidingly received within working channel 385. Conduits 330 can each operably attach to a lumen exiting distal link 365D, lumens 368, such that a tool 400 or camera 800 or the like, which can be slidingly received within the respective working channel 330, for exiting the distal end of probe 300.

In these embodiments, a distal articulation section 360 can be articulated independently of the proximal portion of outer probe 350, for example, when the proximal portion of 350 is in a locked state, and cables 361 are manipulated to articulate the distal section 360.

In some embodiments, one or more steering and or navigating algorithms can be executed by one or more hardware computer processors and stored in a memory of the system 10 to drive probe 300. These algorithms when part of a computer program can actively control the relative position and state of inner probe 310, outer probe 350, and/or distal section 360, to articulate probe 300 in space.

Referring to FIG. 8A, a perspective view of a distal most outer link 355D is illustrated, in accordance with embodiments of the present inventive concepts.

In some embodiments, the outer link 355D comprises an extended link body 356E with one or more recesses 3561. A cable 361 can extend distally, through a proximal lumen 358a, thru recess 3561, and continue through distal lumen 358b, to the distal articulating section 360, e.g., shown in FIG. 7. In some embodiments, the cable 351 extends through a proximal lumen 358a, and terminates within recess 3561, with termination point 3511 (such as a knot or a clip configured to prevent cable 351 from retracting through 358a when pulled). Only one set of four sets of cables is shown but is not limited thereto.

Referring to FIG. 8B, a perspective view of a distal outer link 365D is illustrated, in accordance with embodiments of the present inventive concepts.

A distal link 365D can comprise an extended link body 366E, and one or more recesses 3561. One or more cables 361 (not shown) can terminate in recesses 3561, similar to link 355D as described hereabove in reference to FIG. 8A. Distal link 365D can comprise one or more projections 367, extending distally from link body 366E, surrounding one or more lumens 368. One or more lumens 368 can terminate at the distal end of link body 366E (e.g. not extend through a projection 367). In these embodiments, a tool 400 (such as camera 800) can exit a lumen 368 within which it is slidingly received proximal to other tools 400 received within lumens extending through projections 367.

Referring to FIG. 8C, a perspective view of a distal inner link 315D is illustrated, in accordance with embodiments of the present inventive concepts.

In some embodiments, distal inner link 315D includes an extended link body 316E, with a recess defining a space, 319. Cables 311 can extend through lumens 317 and terminate within 319, with termination point 3111, for example, a knot or a clip configured to prevent the cable 311 from retracting through the corresponding lumen 317 when pulled. In some embodiments, termination point 3111 is free to rotate within space 319, such that when cables 311 are de-tensioned as described herein, cables 311 can “unwind” to lessen torsional stress on inner probe 310. For example, while inner probe 310 is manipulated, cables 311 can twist in an undesired manner and exert unwanted torsional forces on inner probe 310. Only one of four of cables shown but is not limited thereto.

Referring to FIG. 9, a perspective view of a probe assembly 300 is illustrated, in accordance with embodiments of the present inventive concepts. Referring additionally to FIGS. 9A-B, perspective views from the underside of a probe assembly 300, with a cart 320 in advanced and retracted positions are illustrated, respectively, in accordance with embodiments of the present inventive concepts.

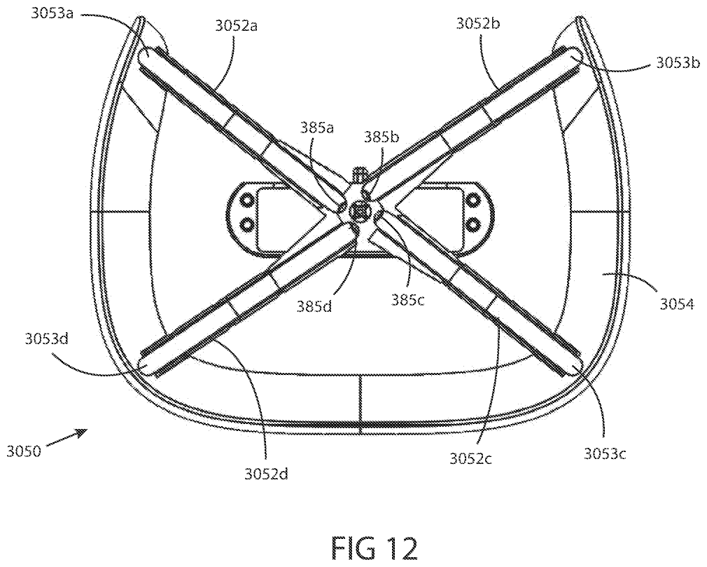

The probe 300 includes a manipulation assembly 3000 that can be operably attached to a proximal end of the outer probe 350 and inner probe 310 (which is positioned within outer probe 350 and not visible in FIGS. 9 and 9A, but shown in FIG. 9B). In some embodiments, the probe 300 includes a support assembly 3050 that at least partially surrounds the manipulation assembly 3000, and comprises a radial support 3054, fixedly attached to one or more channels 3052, connected to manipulation assembly 3000. The support assembly 3050 is described herebelow in reference to FIG. 12.

In some embodiments, the manipulation assembly 3000 comprises a casing 3010, surrounding one or more components constructed and arranged to manipulate probes 350 and 310, as described below. Casing 3010 can comprise a chamber 3012 which slidingly receives a cart 320, operably attached to the proximal end of probe 310. The cart 320 can comprise a cover 3201, comprising one or more openings such as to provide access to one or more bobbins 326 within cart 320. The cover 3201 can comprise one or more projections 3202 for operably engaging a frame 1511 of the cart 151.

The casing 3010 can comprise a cover 3011, which in turn comprises one or more openings such as to provide access to one or more bobbins 376 within the casing 3010. The cover 3011 can comprise one or more recesses 3014 for operably engaging one or more alignment pins 1273 of 170.

Referring to FIG. 10, a perspective view from the underside of a manipulation assembly 3000 with some components removed, including covers 3201 and 3011 and two bobbins 376, is illustrated, in accordance with embodiments of the present inventive concepts.

In some embodiments, the inner probe 310 includes a proximal shaft 3151 slidingly received within at least the proximal portion of the outer probe 350. The proximal shaft 3151 can comprise an elongate body with one or more channels therethrough, of similar but elongated construction to links 315. The support assembly 3050 can comprise a central passageway 3056 to allow the proximal shaft 3151 to translate therethrough and into the probe 350. In some embodiments, the shaft 3151 is operably attached to the cart 320, housing multiple bobbins 326, each rotatably positioned on an axle 3161. The cart 320 can operably and removably attach to probe translation assembly 150 described herein, with capstans 156 operably engaging a set of bobbins 326. The capstans 156 can be constructed and arranged to rotate bobbins 326, thereby tensioning and de-tensioning cables 311, wound about bobbins 326, and extending throughout probe 310. The translation of the probe translation assembly 150, relative to the probe support assembly 170 can cause the translation of cart 320, relative to manipulation assembly 3000, driving the inner probe 310 within outer probe 350.

The casing 3010 can house multiple bobbins 376, each rotatably positioned on an axle 3571. The manipulation assembly 3000 can operably and removably attach to probe support assembly 170 described herein, with capstans 176 operably engaging bobbins 376. The capstans 176 rotate the bobbins 376, tensioning and de-tensioning cables 351, wound about bobbins 376, and extending throughout probe 350. A translation of the carriage 125, relative to the base 121, can cause the translation of manipulation assembly 3000, driving both probes 350 and 310. In some embodiments, the probe translation assembly 150 is driven “backwards” as the carriage 125 is driven “forwards”, such that the outer probe 350 translates forward relative to a “stationary” inner probe 310.

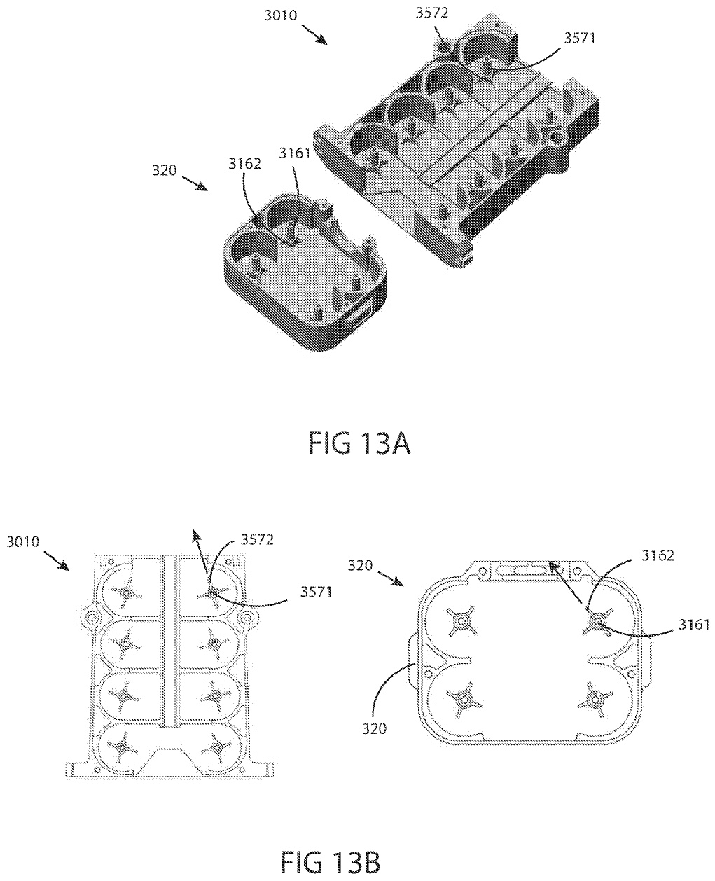

In some embodiments, manipulation assembly 3000 can comprise a rear housing lower portion 3801 and a rear housing upper portion 3802. The lower portion 3801 and the upper portion 3802 can form one or more guide slots 3803 (e.g. lower portion 3801 and the upper portion 3802 can be assembled and/or manufactured to form guide slot 3803). In some embodiments, an external guide 3804 (as shown in FIG. 13B) is constructed and arranged to slide inside a guide slot 3803. External guide 3804 can comprise a projection that extends from a side of cart 320. Guide slot 3803 can be configured to retain cart 320 inside the manipulation assembly 3000 prior to installation on manipulation assembly 120.

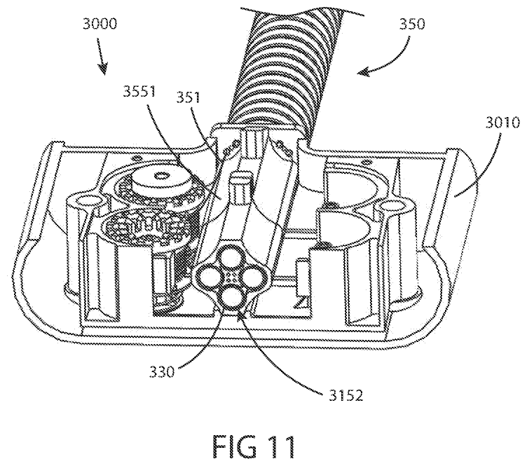

Referring to FIGS. 11 and 12, sectional views of portions of a manipulation assembly 3000 are illustrated, in accordance with embodiments of the present inventive concepts.

In some embodiments, the outer probe 350 includes a proximal shaft 3551 operably attached to the casing 3010. The proximal shaft 3551 can comprise an elongate body, and include one or more channels 3152 aligned with channels 385 of probe assembly 300 described herein. Lumens 3053 can extend through conduits 3052 of the support assembly 3050, and each can align with a channel 3152 (shown in FIG. 11). In some embodiments, one or more working conduits 330 extend proximally from channels 385 through lumens 3053. A tool 400 inserted into lumen 3053 is guided through the conduit 3052 into the working channel 385 and/or working conduit 330.

As shown in FIG. 12, the proximal ends of conduits 3052 can be oriented in an “X” like pattern, such that the proximal ends of conduits 3052 align with tools 400 extending from tool drives 200, as described in reference to FIG. 1. The channels 385 can be oriented in a “T” like pattern (e.g. rotated 45 from the pattern of the proximal ends of conduits 3052). The support assembly 3050 can comprise a “twist” along at least a portion of its length, such that a conduit 3053a is aligned at its proximal end with a tool drive 200, and at its distal end with a working channel 385a, as shown.

Referring to FIGS. 13A and B, perspective and bottom views of portions of a casing 3010 and a cart 320, with bobbins 376 and 326 removed, are illustrated, in accordance with embodiments of the present inventive concepts.

As shown, each axle 3571,3161 comprises one or more support fins 3572, 3162. Each axle 3571,3161 can comprise four fins separated by 900 in the pattern shown, but not limited thereto. The pattern of the fins 3572, 3162 can be oriented in the direction of force applied by bobbins to axles 3571,3161 (as shown by the arrows in FIG. 13B), such as when cables wound about the bobbins are under tension. This orientation can be optimized to strengthen axles 3571, 3161 against the most likely direction of force applied, and tailored to each axle 3571,3161 based on its location and the direction of the force applied.

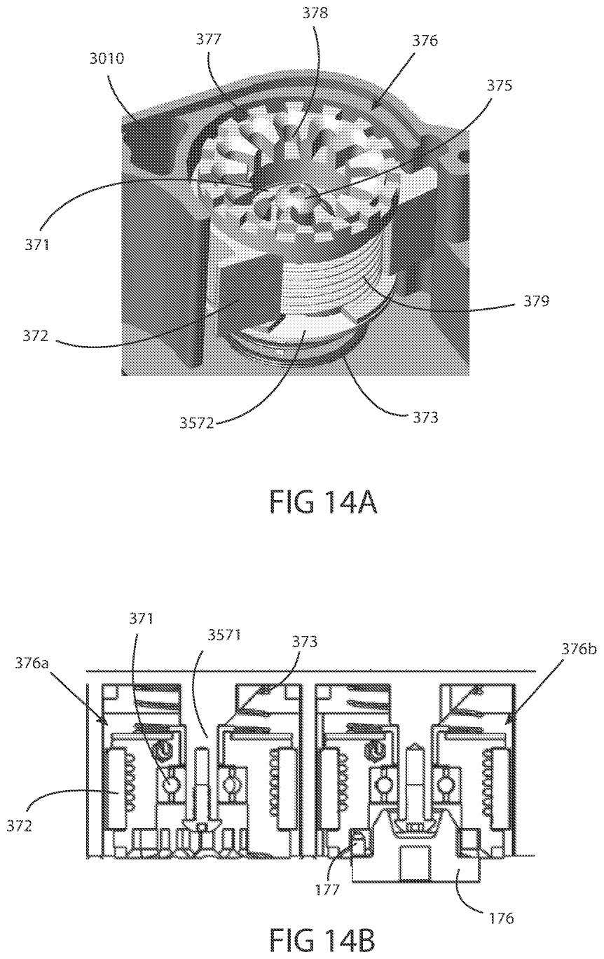

Referring to FIGS. 14A and B, a perspective view of a bobbin 376 operably attached to an axle 3571 of a casing 3010 and a sectional view of two bobbins 376 are illustrated, in accordance with embodiments of the present inventive concepts. In FIG. 14B, a bobbin 376B is shown engaged with a capstan 176.

In some embodiments, one or more bobbins 376 comprise spiral grooves 379 for receiving a wound cable, such as a cable 351 (not shown). Each bobbin 376 can include a clip 372 for maintaining a cable 351 within the bobbin's grooves 379. Clip 372 can engage a groove or other feature of a surrounding housing to prevent 372 from rotating with bobbin 376. A bearing 371 can be positioned between the bobbin 376 and axle 3571. A spring 373 can oppose a washer 3572, which opposes the bobbin 376, biasing the bobbin 376 down (as shown in FIG. 14B). Each bobbin 376 can comprise a castellated portion 377 configured to engage a castellated portion of cover 3011 (not shown), but configured to prevent unwanted rotation of the bobbin 376. A screw 375 can prevent the bobbin 376 from disengaging the axle 3571.

A bobbin 376 can comprise one or more recesses 378, configured to slidingly receive one or more projections 177 of capstan 176, to rotatably engage the two. The capstan 176 can be configured to depress the bobbin 376 against a spring 373, disengaging the castellated portion 377 from the cover 3011, and allowing the bobbin 376 to be rotated by the capstan 176. The bobbins 326 can be constructed and arranged similarly.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for performing a medical procedure on a patient, comprising:
an articulating probe assembly, comprising:
an inner probe comprising multiple articulating inner links;
an outer probe surrounding some or all of the inner probe and comprising multiple articulating outer links; and
at least two working channels that exit a distal portion of the probe assembly;
at least one tool configured to translate through one of the at least two working channels; and
a feeder for controlling the articulating probe assembly.

2.-13. (canceled)
